(19) United States
(12) Patent Application Publication
(10) Pub. No.: US 2017/0188806 A1
(43) Pub. Date: Jul. 6, 2017

(54) SYSTEM AND METHOD FOR IDENTIFYING OR ALERTING AN APPROPRIATE EYE CARE PROVIDER

(71) Applicant: Ram Peddada, Dothan, AL (US)

(72) Inventor: Ram Peddada, Dothan, AL (US)

(21) Appl. No.: 15/271,317

(22) Filed: Sep. 21, 2016

Related U.S. Application Data
(63) Continuation-in-part of application No. 14/986,681, filed on Jan. 2, 2016.

(51) Int. Cl. A61B 3/00; G06F 19/00

(52) U.S. Cl. CPC A61B 3/0025; A61B 3/0033; G06F 19/326; G06F 19/322; H04L 67/42

(57) ABSTRACT

A system and method to identify and track eye-related symptoms are presented. A method of identifying an appropriate eye-care provider or to alert an established provider of new eye-related symptoms based on a user's experienced symptoms are disclosed. Based on a user's experienced symptoms, the system may provide for identifying an appropriate eye-care provider by providing the field of medicine, subfield of medicine, individual eye-care providers, or alert information relating to the user's experienced symptoms. The system may enable users or eye-care providers to track user symptoms as a means of determining whether prompt treatment of such symptoms is necessary. The system may assess a user's experienced symptoms and inform a user or an eye-care provider of whether the user needs prompt treatment.

### CROSS REFERENCES

This application is a continuation-in-part of prior U.S. application Ser. No. 14/986,681, filed on Jan. 2, 2016, currently pending, which application is incorporated herein by reference.

### FIELD OF THE INVENTION

A preferred implementation of a version of the present disclosure refers to a system and method for determining and tracking changes in eye-related symptoms to identify an appropriate new eye-care provider or to alert an established eye-care provider.

### BACKGROUND

In an age where information is seemingly readily available on the internet, an opportunity exists for individuals to obtain medical knowledge concerning their eyes. However, what is lacking is a mechanism by which this knowledge is customized to the specific symptoms and signs of the individuals—not the way the individual interprets the pictures or language on an informational web site—but the way an individual's body, in this case the eye, is functioning or failing. Depending on the underlying cause of an individual's experienced symptoms, these symptoms can be indicative of a fairly minor condition, such as dry eyes, or of a more serious condition, such as age-related macular degeneration. These, in turn, determine what type of eye-care provider the individual requires. This is relevant since there are many different specialists within the “eye care” field performing mutually exclusive functions, from a non-invasive check for glasses performed by an optometrist to a complex surgical procedure of a retinal surgery performed by a vitreoretinal specialist. As a result, individuals self-assessing their symptoms may misdiagnose their symptoms and seek out an eye-care provider inappropriate for their needs. Thus, the use of online resources that provide information not tailored to the specific functioning of an individual eye in terms of identifying an appropriate eye-care provider is generally unhelpful and often delays the time in which an appropriate eye-care provider is identified, thus allowing an individual's condition to worsen.

Moreover, when individuals immediately seek out an eye-care provider without first identifying an appropriate eye-care provider, the eye-care provider initially sought out may be inappropriate for their needs. Accordingly, this approach can lead to multiple eye-care provider visits and result in greater expenses and waste valuable time, thus allowing an individual's condition to worsen before an appropriate eye-care provider is identified.

While there are online resources that attempt to provide diagnosis based on a user's symptoms, such resources merely provide a list of possible conditions in which an individual may be suffering from without indicating an appropriate eye-care provider. Accordingly, these resources also frequently result in an individual being subjected to multiple eye-care provider visits before identifying an appropriate eye-care provider. Moreover, while there are online resources that identify local eye-care providers, such resources do not provide a list of appropriate eye-care providers based on an individual's unique symptoms or tailors the list of eye-care providers to the preferences of the individual, which, again, results in multiple visits to eye-care providers and wastes precious time.

Accordingly, there is a need for a system and method for immediate, remote triaging that allows a user to identify an appropriate eye-care provider based on the individual's experienced symptoms. Furthermore, there is a need for a system and method that allows an individual to identify an eye-care provider based on both the individual's experienced physical symptoms as well as personal preferences selected by the individual.

### SUMMARY

In accordance with one aspect of the principles of the present disclosure, a system and method for identifying an appropriate eye-care provider are provided. In one preferred embodiment, the system of the present disclosure stores a wide array of data within the system's database, wherein such data may include: vision-related symptom data, question and possible answer data, eye-care provider data, field of medicine data, subfield of medicine data, test result data, and test data. To interact with the system and conduct the method disclosed herein a user first makes a request to a server to access the vision-related symptom and test result data stored within the system's database. The vision-related symptom data within the database has a series or question and possible answer data tied thereto and the test result data within the database has a series of tests tied thereto. Based on a user's request, a server then receives, communicates, generates, and provides a series of questions and tests to the user through a user interface. Through a user interface, the system then allows a user to input answers to the questions and conduct the tests provided to the user via a server and user interface. Upon answering the questions and conducting the tests presented, a server within the system then receives the user-generated answers and test results. A processor or server then processes the user-generated answers and test results to establish a statistical correlation between the user-generated answers and test results and the vision-related symptom and test results data stored within the system's database. Based on the presence of a statistical correlation, a server then communicates, generates, and provides to the user as output information, zero or more fields or subfields of medicine that are tied to the vision-related symptoms and test results that have a statistical correlation to the answers and test results generated by the user.

In one preferred embodiment, eye-care provider data containing information about specific eye-care providers is also tied to the vision-related symptom data and test result data within the database such that the eye-care provider data will be included within the output information communicated, generated, and provided by a server. In another preferred embodiment, alert data is also tied to the vision-related symptom and test result data within the system's database in the same or similar fashion.

In one preferred embodiment, the system contains one or more filtering tools that may be utilized by a user or system administrator to further limit the output information provided to a user by a server. Such filtering tools may permit a user or system administrator to limit the output information provided to a user based on distance from the user's location to a specific eye-care provider, geographic parameters associated with a specific eye-care provider, or whether an eye-care provider is open on Saturdays or provides other special accommodations for patients. In another embodiment, the system and method are accessible to a user via a mobile application. Yet another embodiment of the present disclosure provides for the output information provided by a system server being delivered to the user via email. In another preferred embodiment, the system includes an eye-care provider interface that enables eye-care providers to access and manipulate eye-care provider data within the system's database based on a permission level assigned to an eye-care provider.

Additionally, in one preferred embodiment, patients with progressing pre-existing disease needing periodic treatment may use this system of the present disclosure to assess their status objectively from time to time and be more certain of the need to travel to the care facility, especially if their caregiver is able to evaluate and confirm the changes remotely.

Additional features and advantages of the present disclosure will be set forth in the description which follows, and will be apparent from the description, or may be learned by practice of the present disclosure. The foregoing general description and following detailed description are exemplary and explanatory and are intended to provide further explanation of the present disclosure.

### DESCRIPTION OF THE DRAWINGS

These and other features, aspects, and advantages of the present disclosure will become better understood with regard to the following description, appended claims, and accompanying drawings where:

FIG. 1 is a diagram of an example environment in which techniques described herein may be implemented;

FIG. 2 is an exemplary diagram of a client of FIG. 1 according to an implementation consistent with the principles of the present disclosure;

FIG. 3 is a diagram of an example of a computing device and mobile computing device;

FIG. 4 is a diagram illustrating an example system configuration and process according to an implementation consistent with the principles of the present disclosure;

FIG. 5 is a diagram illustrating an example system configuration and process according to an implementation consistent with the principles of the present disclosure;

FIG. 6 is a diagram illustrating an example system configuration according to an implementation consistent with the principles of the present disclosure;

FIG. 7 is a diagram illustrating an example system configuration according to an implementation consistent with the principles of the present disclosure;

FIG. 8 illustrates an example of a vision-related test for the user to conduct via a user interface;

FIG. 9 illustrates an example of a vision-related test for the user to conduct via a user interface;

FIG. 10 illustrates an example of a vision-related test for the user to conduct via a user interface;

FIG. 11 illustrates an example of a vision-related test for the user to conduct via a user interface; and

FIG. 12 is a diagram illustrating an example system configuration according to an implementation consistent with the principles of the present disclosure.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises” and grammatical equivalents thereof are used herein to mean that other components, steps, etc. are optionally present. For example, a system “comprising” components A, B, and C can contain only components A, B, and C, or can contain not only components A, B, and C, but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

Systems and methods consistent with the principles of the present disclosure may provide solutions for efficiently identifying appropriate eye-care providers, specialists, physicians, or any individual or organization involved in the treatment of vision-related symptoms based on the symptoms suffered by an individual. For example, the systems and methods of the present disclosure may permit a user to identify one or more eye-care providers based on the user's unique symptoms.

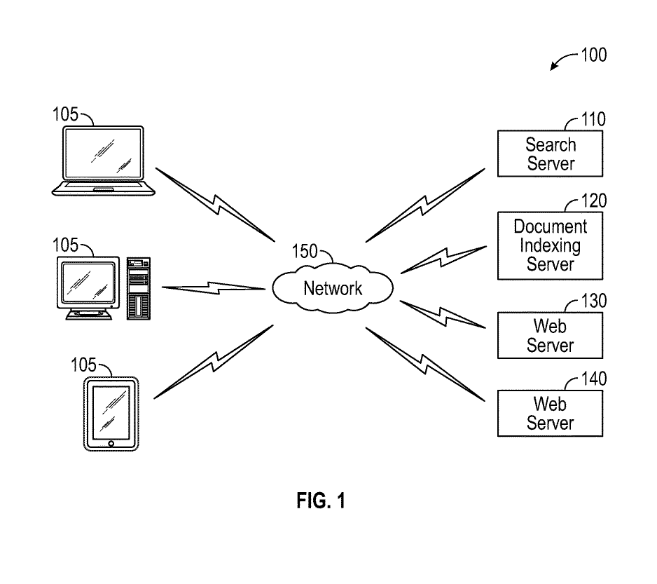

FIG. 1 is a diagram of an example environment 100 in which techniques described herein may be implemented. Environment 100 may include multiple clients 105 connected to one or more servers 110-140 via a network 150. In some implementations, and as illustrated, server 110 may be a search server, that may implement a search engine; and server 120 may be a document indexing server, e.g., a web crawler; and servers 130 and 140 may be general web servers, such as servers that provide content to clients 105. Clients 105 and servers 110-140 may be connected to network 150 via wired, wireless, or a combination of wired and wireless connections.

Three clients 105 and four servers 110-140 are illustrated as connected to network 150 for simplicity. In practice, there may be additional or fewer clients and servers. Also, in some instances, a client may perform the functions of a server and a server may perform the functions of a client.

Clients 105 may include devices of users that access servers 110-140. A client 105 may include, for instance, a personal computer, a wireless telephone, a personal digital assistant (PDA), a laptop, a smart phone, a tablet computer, or another type of computation or communication device. Servers 110-140 may include devices that access, fetch, aggregate, process, search, provide, and/or maintain documents. Although shown as single components 110, 120, 130, and 140 in FIG. 1, each server 110-140 may, in some implementations, be implemented as multiple computing devices, which potentially may be geographically distributed.

Search server 110 may include one or more computing devices designed to implement a search engine, such as a documents/records search engine, general webpage search engine, etc. Search server 110 may, for example, include one or more web servers to receive search queries and/or inputs from clients 105, search one or more databases in response to the search queries and/or inputs, and provide documents or information, relevant to the search queries and/or inputs, to clients 105. In some implementations, search server 110 may include a web search server that may provide webpages to clients 105, where a provided webpage may include a reference to a web server, such as one of web servers 130 or 140, at which the desired information and/or links is located. The references, to the web server at which the desired information is located, may be included in a frame and/or text box, or as a link to the desired information/document.

Document indexing server 120 may include one or more computing devices designed to index documents available through network 150. Document indexing server 120 may access other servers, such as web servers that host content, to index the content. In some implementations, document indexing server 120 may index documents/records stored by other servers, such as web servers 130 and 140 and, connected to network 150. Document indexing server 120 may, for example, store and index content, information, and documents relating to eye-care providers, fields and subfields of medicine associated with vision-related symptoms, information pertaining to the severity of conditions associated with vision-related symptoms, and vision-related tests.

Web servers 130 and 140 may each include web servers that provide webpages to clients. The webpages may be, for example, HTML-based webpages. A web server 130/140 may host one or more websites. A website, as the term is used herein, may refer to a collection of related webpages. Frequently, a website may be associated with a single domain name, although some websites may potentially encompass more than one domain name. The concepts described herein may be applied on a per-website basis. Alternatively, in some implementations, the concepts described herein may be applied on a per-webpage basis.

While servers 110-140 are shown as separate entities, it may be possible for one or more servers 110-140 to perform one or more of the functions of another one or more of servers 110-140. For example, it may be possible that two or more of servers 110-140 are implemented as a single server. It may also be possible for one of servers 110-140 to be implemented as multiple, possibly distributed, computing devices.

Network 150 may include one or more networks of any kind, including, but not limited to, a local area network (LAN), a wide area network (WAN), a telephone network, such as the Public Switched Telephone Network (PSTN), an intranet, the Internet, a memory device, another type of network, or a combination of networks.

Although FIG. 1 shows example components of environment 100, in other implementations, environment 100 may contain fewer components, different components, differently arranged components, and/or additional components than those depicted in FIG. 1. Alternatively, or additionally, one or more components of environment 100 may perform one or more other tasks described as being performed by one or more other components of environment 200.

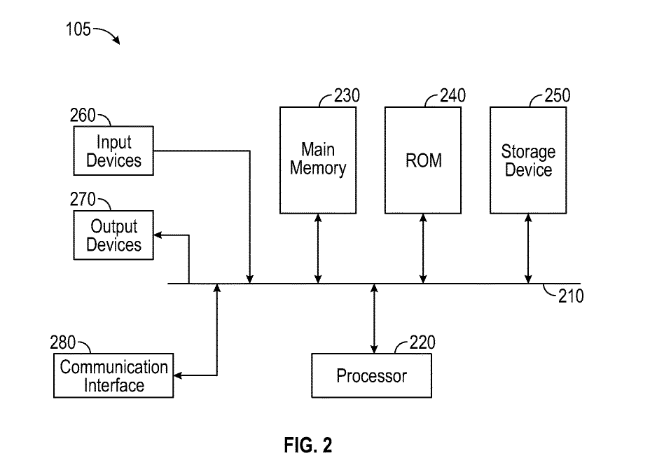

FIG. 2 is an exemplary diagram of a user/client 105 or server entity (hereinafter called “client/server entity”), which may correspond to one or more of the clients and servers, according to an implementation consistent with the principles of the invention. The client/server entity 105 may include a bus 210, a processor 220, a main memory 230, a read only memory (ROM) 240, a storage device 250, one or more input devices 260, one or more output devices 270, and a communication interface 280. Bus 210 may include one or more conductors that permit communication among the components of the client/server entity 105.

Processor 220 may include any type of conventional processor or microprocessor that interprets and executes instructions. Main memory 230 may include a random access memory (RAM) or another type of dynamic storage device that stores information and instructions for execution by processor 220. ROM 240 may include a conventional ROM device or another type of static storage device that stores static information and instructions for use by processor 220. Storage device 250 may include a magnetic and/or optical recording medium and its corresponding drive.

Input device(s) 260 may include one or more conventional mechanisms that permit an operator to input information to the client/server entity 105, such as a scanner, phone, camera, scanning device, keyboard, a mouse, a pen, voice recognition and/or biometric mechanisms, etc. Output device(s) 270 may include one or more conventional mechanisms that output information to the operator, including a display, a printer, a speaker, an alarm, a projector, etc. Communication interface 280 may include any transceiver-like mechanism that enables the client/server entity 105 to communicate with other devices 105 and/or systems. For example, communication interface 280 may include mechanisms for communicating with another device 105 or system via a network, such as network 150.

As will be described in detail below, the client/server entity 105, consistent with the principles of the invention, performs certain receiving, communicating, generating, output providing, correlating, and storing operations. The client/server entity 105 may perform these operations in response to processor 220 executing software instructions contained in a computer-readable medium, such as memory 230. A computer-readable medium may be defined as one or more physical or logical memory devices and/or carrier waves.

The software instructions may be read into memory 230 from another computer-readable medium, such as data storage device 250, or from another device via communication interface 280. The software instructions contained in memory 230 causes processor 220 to perform processes that will be described later. Alternatively, hardwired circuitry may be used in place of or in combination with software instructions to implement processes consistent with the principles of the invention. Thus, implementations consistent with the principles of the invention are not limited to any specific combination of hardware circuitry and software.

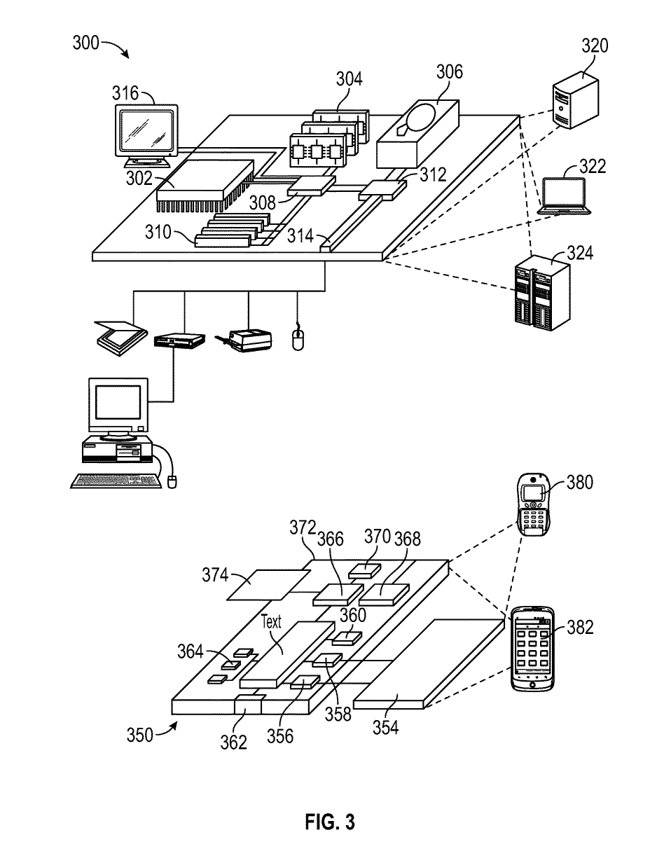

FIG. 3 is a diagram of an example of a computing device 300 and a mobile computing device 350, which may be used with the techniques described here. Computing device 300 or mobile computing device 350 may correspond to, for example, a client 205 and or a server 210-240. Computing device 300 is intended to represent various forms of digital computers, such as laptops, desktops, workstations, personal digital assistants, servers, mainframes, and other appropriate computers. Mobile computing device 350 is intended to represent various forms of mobile devices, such as scanners, scanning devices, personal digital assistants, cellular telephones, smart phones, tablet computers, and other similar computing devices. The components show in FIG. 3, their connections and relationships, and their functions, are meant to be examples only, and are not meant to limit implementations described herein.

Computing device 300 may include a processor 302, a memory 304, a storage device 306, a high-speed interface 308 connecting to a memory 304 and high-speed expansion ports 310, and a low-speed interface 312 connecting to a low-speed expansion port 314 and a storage device 306. Each of components 302, 304, 306, 308, 310, 312, and 314 are interconnected using various buses, and may be mounted on a common motherboard or in other manners as appropriate. Processor 302 can process instructions for execution within computing device 300, including instructions stored in memory 304 or on storage device 306 to display graphical information for a graphical user interface (GUI) on an external input/output device, such as display 316 coupled to high-speed interface 308. In other implementations, multiple processors and/or multiple buses may be used, as appropriate, along with multiple memories and types of memory. Also, multiple computing devices 300 may be connected, with each device providing portions of the necessary operations, as a server bank, a group of blade servers, or a multi-processor system, etc.

Memory 304 stores information within computing device 300. In some implementations, memory 304 includes a volatile memory unit or units. In another implementation, memory 304 may include a non-volatile memory unit or units. Memory 304 may also be another form of computer-readable medium, such as a magnetic or optical disk. A computer-readable medium may refer to a non-transitory memory device. A memory device may refer to storage space within a single storage device or spread across multiple storage devices.

Storage device 306 is capable of providing mass storage for computing device 300. In some implementations, storage device 306 may be or contain a computer-readable medium, such as a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described herein. The information carrier is a computer or machine-readable medium, such as memory 304, storage device 306, or a memory on processor 302.

High-speed interface 308 manages bandwidth-intensive operations for computing device 300, while low-speed interface 312 manages lower bandwidth-intensive operations. Such allocation of functions is an example only. In some implementations, high-speed interface 308 is coupled to memory 304, display 316, such as through a graphics processor or accelerator, and to high-speed expansion ports 310, which may accept various expansion cards. In this implementation, low-speed interface 312 may be coupled to storage device 306 and low-speed expansion port 314. Low-speed expansion port 314, which may include various communication ports, such as USB, Bluetooth, Ethernet, wireless Ethernet, etc., may be coupled to one or more input/output devices, such as a keyboard, a pointing device, a scanner, or a networking device such as switch or router, e.g., through a network adapter.

Computing device 300 may be implemented in a number of different forms, as shown in the figures. For example, computing device 300 may be implemented as a standard server 320, or in a group of such servers. Computing device 300 may also be implemented as part of a rack server system 324. In addition, computing device 300 may be implemented in a personal computer, such as a laptop computer 322. Alternatively, components from computing device 300 may be combined with other components in a mobile device, such as mobile computing device 350. Each of such devices may contain one or more computing devices 300, 350, and an entire system may be made up of multiple computing devices 300, 350 communicating with each other.

Mobile computing device 350 may include a processor 352, a memory 364, an input/output (“I/O”) device, such as a display 354, a communication interface 366, and a transceiver 368, among other components. Mobile computing device 350 may also be provided with a storage device, such as a micro-drive or other device, to provide additional storage. Each of the components 352, 364, 354, 366, and 368 are interconnected using various buses, and several of the components may be mounted on a common motherboard or in other manners as appropriate.

Processor 352 can execute instructions within mobile computing device 350, including instructions stored in memory 364. Processor 352 may be implemented as a chipset of chips that include separate and multiple analog and digital processors. Processor 352 may provide, for example, for coordination of the other components of mobile computing device 350, such as control of user interfaces, applications run by mobile computing device 350, and wireless communication by mobile computing device 350.

Processor 352 may communicate with a user through control interface 358 and display interface 356 coupled to a display 354. Display 354 may be, for example, a TFT LCD (Thin-Film-Transistor Liquid Crystal Display) or an OLED (Organic Light Emitting Diode) display or other appropriate display technology. Display interface 356 may include appropriate circuitry for driving display 354 to present graphical and other information to a user. Control interface 358 may receive commands from a user and convert the commands for submission to processor 352. In addition, an external interface 362 may be provided in communication with processor 352, so as to enable near area communication of mobile computing device 350 with other devices. External interface 362 may provide, for example, for wired communications in some implementations, or for wireless communication in other implementations, and multiple interfaces may also be used.

Memory 364 stores information within mobile computing device 350. Memory 364 can be implemented as one or more of a computer-readable medium or media, a volatile memory unit or units, or a non-volatile memory unit or units. Expansion memory 374 may also be provided and connected to mobile computing device 350 through expansion interface 372, which may include, for example, a SIMM (Single In Line Memory Module) card interface. Such expansion memory 374 may provide extra storage space for device 350, or may also store applications or other information for mobile computing device 350. Specifically, expansion memory 374 may include instructions to carry out or supplement the processes described herein, and may include secure information also. Thus, for example, expansion memory 374 may be provided as a security module for mobile computing device 350, and may be programmed with instructions that permit secure use of mobile computing device 350. In addition, secure applications may be provided via the SIMM cards, along with additional information, such as placing identifying information on the SIMM card in a non-hackable manner.

Expansion memory 374 may include, for example, flash memory and/or NVRAM memory. In some implementations, a computer program product is tangibly embodied in an information carrier. The computer program product contains instructions that, when executed, perform one or more methods, such as those described herein. The information carrier is a computer- or machine readable-medium, such as memory 364, expansion memory 374, or a memory on processor 352, that may be received, for example, over transceiver 368 or external interface 362.

Mobile computing device 350 may communicate wirelessly through communication interface 366, which may include digital signal processing circuitry where necessary. Communication interface 366 may provide for communications under various modes or protocols, such as GSM voice calls, SMS, EMS or MMS messaging, CDMA, TDMA, PDC, WCDMA, CDMA2000, or GPRS, among others. Such communication may occur, for example, through transceiver 368. In addition, short-range communication may occur, such as using a Bluetooth, WiFi, or other such transceiver. In addition, GPS (Global Positioning System) received module 370 may provide additional navigation- and location-related wireless data to mobile computing device 350, which may be used as appropriate by applications running on mobile computing device 350.

Mobile computing device 350 may also communicate audibly using audio codec 360, which may receive spoken information from a user and covert the received spoken information to digital information. Audio codec 360 may likewise generate audible sound for a user, such as through a speaker, e.g., in a handset of mobile computing device 350. Such sound may include sound from voice telephone calls, may include recorded sound, such as voice messages, music files, etc., and may also include sound generated by applications operating on mobile computing device 350.

Mobile computing device 350 may be implemented in a number of different forms, as shown in the figure. For example, mobile computing device 350 may be implemented as a cellular telephone 380. Mobile computing device 350 may also be implemented as part of a smart phone 382, personal digital assistant, or other similar mobile device.

Various implementations described herein can be realized in digital electronic circuitry, integrated circuitry, specially designed ASICs (application specific integrated circuits), computer hardware, firmware, software, and/or combinations thereof. These various implementations can include implementations in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs, also known as programs, software, software applications, or code, include machine instructions for a programmable processor, and can be implemented in a high-level procedural and/or object-oriented programming language, and/or in assembly/machine language. As used herein, the terms “machine-readable medium” and “computer-readable medium” refer to any apparatus and/or device, such as magnetic discs, optical disks, memory, Programmable Logic Devices (“PLDs”), used to provide machine instructions and/or data to a programmable processor, including a machine-readable medium that receives machine instructions as a machine-readable signal. The term “machine-readable signal” refers to any signal used to provide machine instructions and/or data to a programmable processor.

The contents of computer-readable medium may physically reside in one or more memory devices accessible by server. Computer-readable medium may include a database of entries corresponding to questions and possible answers related to a particular symptom, tests related to one or more tests results, a field of medicine in which a particular symptom relates, a subfield of medicine in which a particular symptom relates, eye-care provider information related to a particular symptom, and alert data related to a particular symptom. A user or organization's information may be provided in information fields and stored in a database, as set forth herein. Said fields are customizable and may include additional or alternative fields based on the user's needs. Said information is accessible through the server.

To provide for interaction with a user, the techniques described herein can be implemented on a computer having a display device, such as a CRT (cathode ray tube), LCD (liquid crystal display), or LED (Light Emitting Diode) monitor, for displaying information to the user and a keyboard and a pointing device by which the user can provide input to the computer. Other kinds of devices can be used to provide for interaction with a user as well; for example, feedback provided to the user can be any form of sensory feedback, such as visual feedback, auditory feedback, or tactile feedback; and input from the user can be received in any form, including acoustic, speech, or tactile input.

The techniques described herein can be implemented in a computing system that includes a back end component, such as a data server, or that includes a middleware component, such as an application server, or that includes a front end component, such as a client computer having a graphical user interface or Web browser through which a user can interact with an implementation of the techniques described here, or any combination of such back end, middleware, or front end components. The components of the system can be interconnected by any form of medium of digital communication.

A preferred implementation of the present disclosure discloses a system and method for identifying an appropriate eye-care provider based on the symptoms experienced and unique to a user. In a preferred embodiment, the system compares user symptoms to a series of symptoms and test result data stored within the system's database to generate a statistical correlation, and, based on such statistical correlation, generates output information indicating various informational fields relating to appropriate eye-care providers for a user's specific needs. The system comprises a user-interaction system allowing a user to request access to a wide array of data and functionalities embodied within the system and to input user-generated data pertaining to the user's experienced symptoms within the system. The system further comprises a data-assessment system that processes, compares, and correlates user-generated data to the vision-related symptom and test result data stored within the system. The system further comprises a reporting system that retrieves and conveys output information, based on the statistical correlation generated by the data-assessment system, regarding eye-care provider information based on the user's unique needs. In one preferred embodiment, the system further comprises filtering tools to further tailor the output information provided to a user's specific needs and/or preferences.

Consistent with the disclosure set forth herein, the system and method may consist of web and mobile phone versions. In some embodiments, the system may be implemented on a mobile phone application. In these embodiments, a user may utilize a mobile phone to use and interact with the system in a similar fashion as the web-based version of the system disclosed as detailed below. It is understood that, consistent with this disclosure, any device for accessing and utilizing web-based systems or applications may be utilized.

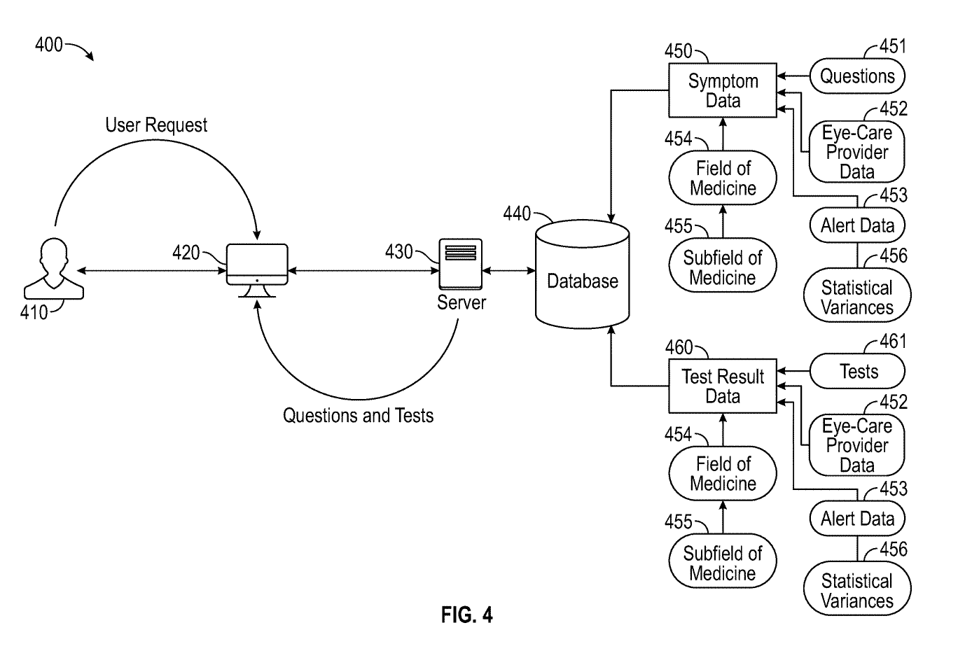

As set forth herein, a preferred implementation of the present disclosure is a method for identifying an appropriate eye-care provider based on a user's experienced symptoms and specific needs. As illustrated in FIG. 4, generally, a series of vision-related symptom data 450 and test result data 460 is stored within a database 440 in the system 400. In one embodiment, the system 400 utilizes a database management system such as Microsoft SQL or similar. Tied to the symptom data 450 is a series of questions and possible answers 451, wherein each question and possible answer corresponds to one or more symptoms within the symptom data 450 contained within the database 440, these questions and possible answers 451 being stored within the database 440 as digital data. Tied to the test result data 460 is one or more tests 461, wherein each test corresponds to one or more test results stored within the test result data 460, the tests being stored within the database 440 as digital data.

In one preferred embodiment the vision-related symptom data 450 and test result data 460 are tied to field of medicine data 454. In this preferred embodiment each vision-related symptom contained within the vision-related symptom data 450 and each test result contained within the test result data 460 that relates to a specific field of medicine within the field of medicine data 454 is grouped together. Within this preferred embodiment, each field of medicine within the field of medicine data 454 has the option of having zero or more vision-related symptoms or test results tied to a particular field of medicine. For example, a field of medicine stored within the field of medicine data 454 may be the field of ophthalmology or oncology, however, it is understood that the data stored within the field of medicine data 455 may contain any field of medicine. Thus, for example, a vision-related symptom or test result which is indicative of brain cancer would be tied to the field of oncology or neurology, whereas a vision-related symptom or test result which is indicative of a retinal disease, such as age-related macular degeneration, would be tied to the field of ophthalmology, etc.

In yet another embodiment, each field of medicine within the field of medicine data 455 has zero or more subfields of medicine that pertain to specific practice areas within a given field of medicine. For example, if the field of medicine is ophthalmology, the subfields of ophthalmologist, optometrist, and retina specialist may be populated within or tied to the ophthalmology field of medicine and conveyed to a user as such, depending on the pertinent vision-related symptom data 450 and test result data 460 to which the field of medicine is tied. In this embodiment, such subfields of medicine are stored as subfield of medicine data 454 within the database 440.

In another preferred embodiment, the database 440 within the system 400 also stores eye-care provider data 452. Such eye-care provider data 452 may either be tied directly to the vision-related symptom data 450 and the test result data 460 directly, as illustrated within FIG. 4, or indirectly tied to the vision-related symptom data 450 and test result data 460. In embodiments wherein the eye-care provider data 452 is tied indirectly to the vision-related symptom data 450 and test result data 460, the eye-care provider data 452 is directly tied to the field of medicine data 454 or subfield of medicine data 455, which is, in turn, tied to the vision-related symptom data 450 and test result data 460. In such indirectly tied embodiments, the eye-care provider data 452 relating to a specific field or subfield of medicine within the field of medicine data 454 or subfield of medicine data are grouped together. In such indirectly tied embodiments, the eye-care provider data 452 contains information regarding individual or organizational eye-care providers within the field of medicine or subfield of medicine to which an eye-care provider within the eye-care provider data 452 is tied.

In embodiments wherein the eye care provider data 452 is directly tied to the vision-related symptom data 450 and test result data 460, the eye-care providers within the eye-care provider data 452 that relate to a specific vision-related symptom within the vision-related symptom data 450 and test result within the test result data 460 are grouped together. In such directly tied embodiments, the eye-care provider data contains information regarding individual or organizational eye-care providers that diagnose, specialize, provide surgical assistance, or otherwise treat or do business relating to the vision-related symptom or test result data in which the eye-care provider data 452 is tied.

The information contained within the eye-care provider data 452 may include, but is not limited to: the names of eye-care providers, the addresses of eye-care providers, the telephone numbers of eye-care providers, the websites of eye-care providers, maps indicating the location of eye-care providers, information indicating whether an eye-care provider is open on Saturdays or provides other special accommodations for patients. The term “eye-care provider” is understood to include, but is not limited to: organizational entities such as large or small businesses which provide eye-diagnostic, surgical, or eye-device services; individual specialists or physicians who specialize in diagnostic assessment or provide treatment within a specific field or subfield of medicine; or any combination or blend therein.

In yet another preferred embodiment, the database 440 of the system 400 also stores a series of alert data 453. In this preferred embodiment, the alert data 453 may be tied to the vision-related symptom data 450 and test result data 460 such that each vision-related symptom contained within the vision-related symptom data 450 and each test result contained within the test result data 460 relates to an alert contained within the alert data 453 and is grouped together therein. Such alert data may include but is not limited to information: indicating the urgency of a user's 410 condition, indicating the severity of a user's 410 condition, indicating the progression of a user's 410 condition, or indicating whether a user 410 needs to promptly see and eye-care provider, based on the user's 410 experienced symptoms. In one preferred embodiment, the alert data 453 is tied to statistical variances 456, wherein each statistical variance is indicative of differences between various eye-related symptoms within the vision-related symptom data 450 and differences between various test results stored within the test result data 460.

When alert data 453 relating to a user's 410 unique condition is outputted to a user interface 420 for the user to view it is understood that the alert data 453 may be depicted in a text-based, image-based, or figure-based form. In some embodiments, for example, if the alert data 453 returned to a user 410, based on the user's 410 experienced symptoms, indicates that the user's 410 symptoms are severe and the user 410 should seek immediate medical assistance such alert information may be conveyed to the user via a user interface 420 as a text-based message. In other embodiments, such alert data 453 may be indicated through the use of an image or figure which indicates the severity of the users of the condition and whether medical assistance is needed. In some embodiments, the alert data 453 may be presented to the user via text or image, where such text or image is color-coded in accordance with the severity of the symptom.

In addition to the question and possible answer data 451, it is understood and contemplated by the present disclosure that other data sets selected form the group of: eye-care provider data 452, alert data 453, field of medicine 454, and subfield of medicine data 455 may be tied to the vision-related symptom data 450 stored in the database 440 of the system 400 in any possible combination. For example, in one preferred embodiment, questions and possible answer data 451, eye-care provider data 452, alert data 453, field of medicine data 454, and subfield of medicine data 455 are all tied to the vision-related symptom data 450 stored within the database 440 system. In another example of one possible embodiment, only the question and possible answer data 451, the field of medicine data 454, and subfield of medicine data 455 may be tied to the vision-related symptom data 450 within the database 440 of the system 400. In yet another example of a possible embodiment, only the question and possible answer data 451 and eye-care provider data 452 may be tied to the vision-related symptom data 450 in the database 440 of the system.

In addition to the test data 461, it is understood and contemplated by the present disclosure that other data sets selected from the group of: eye-care provider data 452, alert data 453, field of medicine 454, and subfield of medicine data 455 may be tied to the test result data 460 stored in the database 440 of the system 400 in any possible combination. For example, in one preferred embodiment, the test data 461, eye-care provider data 452, alert data 453, field of medicine data 454, and subfield of medicine data 455 are all tied to the test result data 460 stored within the database 440 of the system 400. In another example of one possible embodiment, only the test data 461, the field of medicine data 454, and subfield of medicine data 455 are tied to the test result data 460 in the database 440 of the system 400. In yet another example of one possible embodiment of the present disclosure, only the test data 461 and eye-care provider data 452 are tied to the test result data 460 within the database 440 of the system 400.

As further illustrated in FIG. 4, generally, a user 410 utilizes a user interface 420 to access and interact with the data and functionalities stored within the system 400. It is understood that the system 400 may comprise more than one user interface to facilitate more than one user 410 to access and interact with the system 400 at the same time. Through the user interface 420, a user 410 makes an access request to access and compare the vision-related symptoms data 450 and test result data 460 stored within the database 440 of the system 400 to the symptoms being experienced by the user 410.

From the user's 410 access request, a server 430 then receives a series of questions and possible answers, from the questions and possible answers data 451 and at least one vision-related test, from the test data 461 stored within the database 440, where the series of questions and possible answers correlate to vision-related symptoms within the vision-related symptom data 451 and the tests correlate to tests stored within the test result data 461 within the database 440. It is understood that the system 400 may comprise one or more servers 430 to effectuate this method of the present disclosure. Based on the received user 410 access request, a server 430 then communicates the series of questions and possible answers and at least one vision-related test gathered from the database 440. A server 430 then generates the series of questions and possible answers and at least one vision-related test. After such communication, a server 430 then provides the series of questions and possible answers and at least one vision-related test to the user 410 through the user interface 420. In one preferred embodiment of the present disclosure, a server 430 only communicates, generates, and provides to the user 410 a series of questions, without the corresponding possible answers, such that the user must utilize a user interface 420 to personally input answers to the questions presented to a user 410 on a user interface 420.

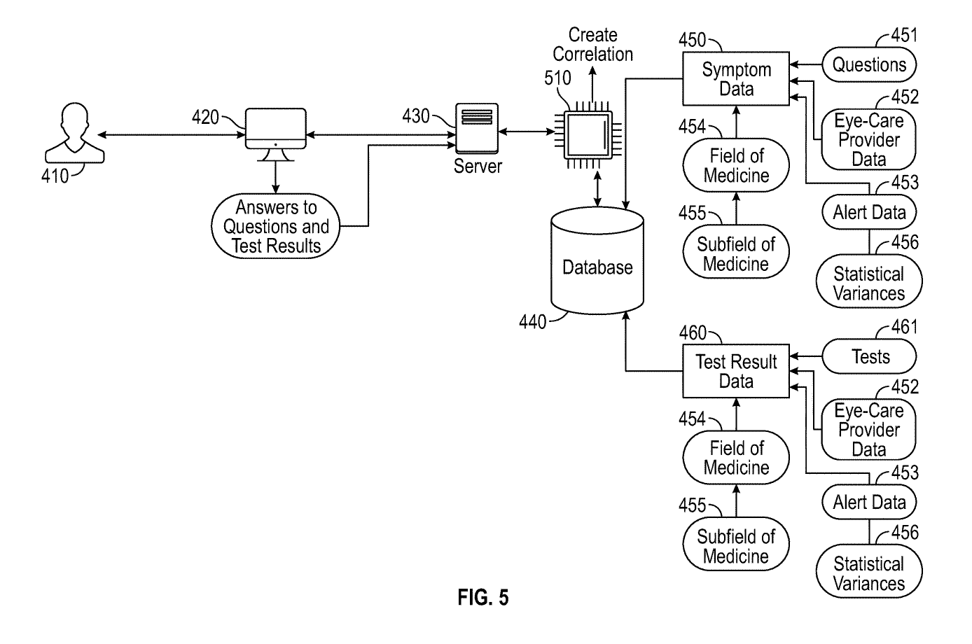

FIG. 5 illustrates a simple representation of how a user 410 interacts with the system 400 once a series of questions and at least one vision-related test has been provided to a user 410 and further illustrates how the system 400 carries out the remaining method steps of the method disclosed herein. As illustrated in FIG. 5, a user 410 utilizes a user interface 420 to answer the questions and conduct the vision-related test presented to the user 410 by the system 400. In one preferred embodiment, in which a server 430 receives, communicates, generates, and provides both question and possible answers, a user 410 answers the questions by clicking or not clicking on one or more key words within the question via a user interface 420, wherein such key words correspond to the answer to the presented question. In another preferred embodiment, where a server 430, receives, communicates, and provides both questions and possible answers, a user 410 answers the questions by clicking on one of one or more possible answers presented along with each question via a user interface 420. In yet another preferred embodiment, where a server 430 receives, communicates, generates, and provides only questions to a user 410, the user answers the questions by manually inputting answers via a user interface 420.

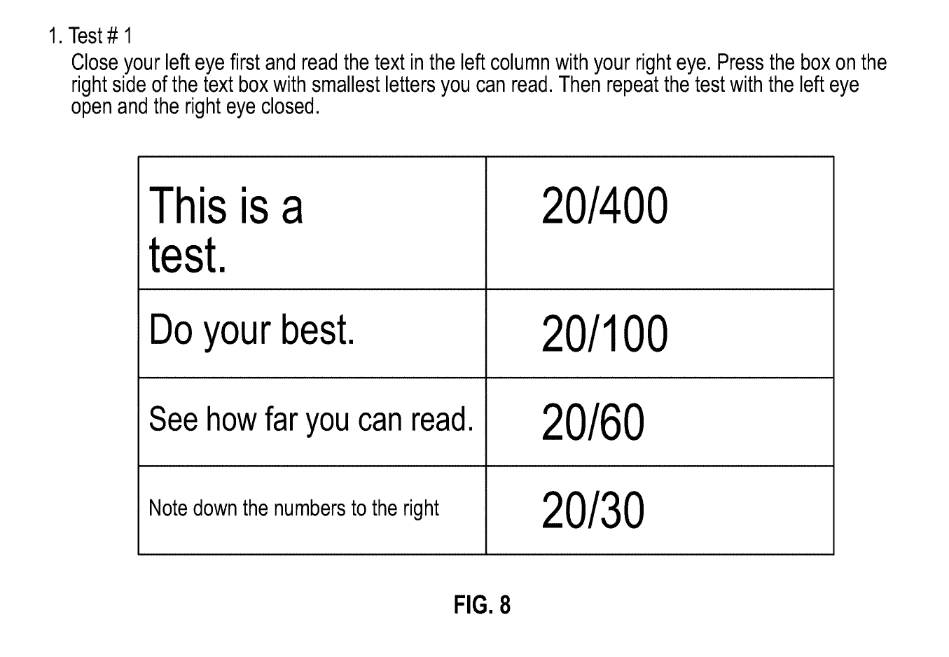

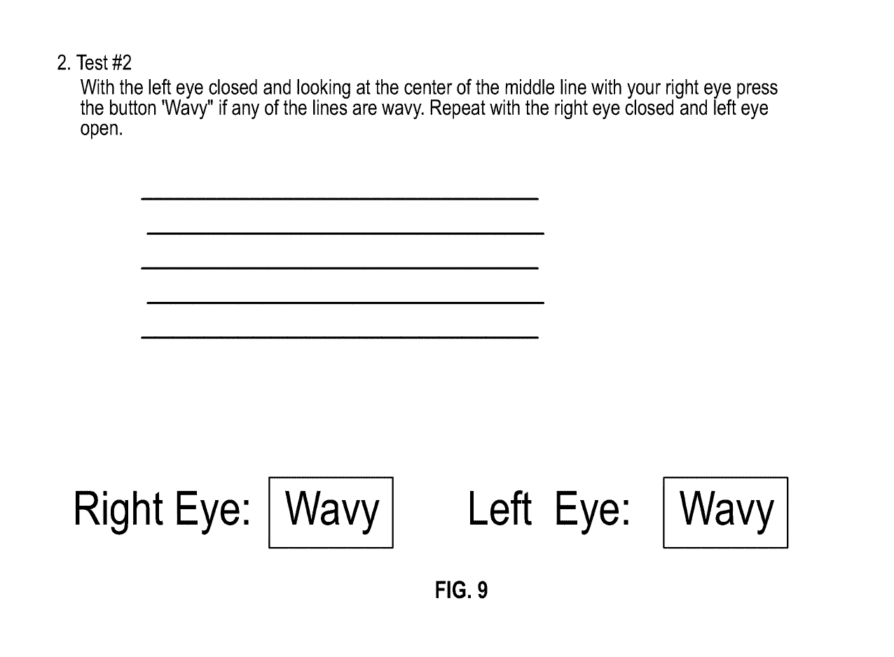

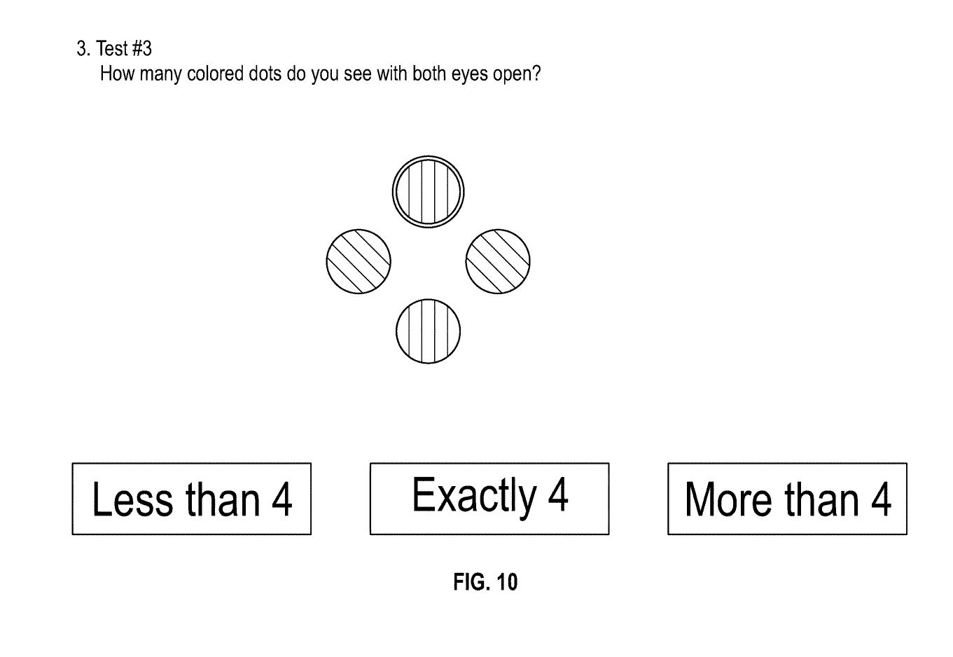

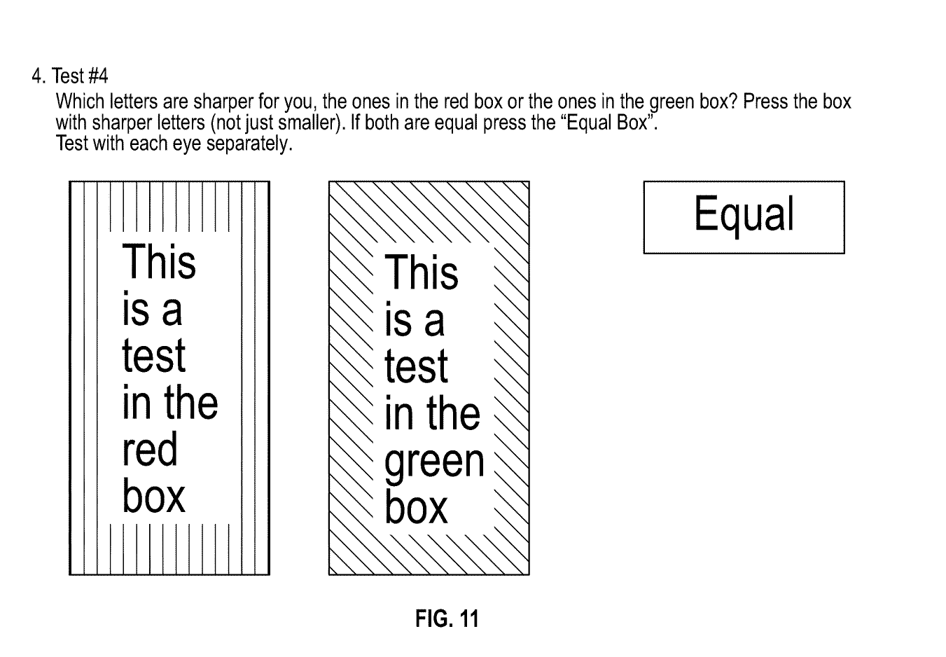

FIGS. 8-11 illustrate examples of possible tests in which a server 430 may receive, communicate, generate, and provide to a user 410 via a user interface 420. The present disclosure contemplates embodiments wherein some tests presented to a user 410 may utilize one or more colors as a part of the test. For instance, the test in FIG. 10 requires a user 410 to select an answer based on the number of colored dots the user 410 can see with both eyes open. Although the coloration of the dots within the test are not shown in FIG. 10, when the test is actually presented to a user 410, in a preferred embodiment, two of the four dots presented would be colored green and the other two dots would be colored red. In one embodiment, the upper and bottom dots would be red and the left and right dots would be green. The colors, number, or orientation of the dates may change and still fall within the scope of the test. The test in FIG. 11 requires a user 410 to choose between two colored boxes based on which box contains letters that appear sharper to the user 410. Although the coloration of the boxes within the test are not shown within FIG. 11, when the test is actually presented to a user 410, in one embodiment, the box containing the words “This is a test in the red box” would be colored red and the box containing the words “This is a test in the green box” would be colored green.

As illustrated within FIGS. 8-11, in one preferred embodiment, a user 410 conducts the vision-related tests provided by selecting one or more possible answer choices accompanying each vision-related test by utilizing a user interface 420. However, in another preferred embodiment of the present disclosure, a user may conduct the vision-related test presented by manually inputting answers to each vision-related test via a user interface 420. It is understood that the tests illustrated in FIGS. 8-11 are examples only and any electronic eye-test may be utilized and the information from the electronic eye-test stored and correlated as set forth herein. As such, vision-related tests utilized by the system may include, but are not limited to, any electronic visual acuity, refraction, visual field, or color vision test. In one preferred embodiment, a test for assessing central and paracentral vision defects and tracking changes in central and paracentral vision disclosed within an application, U.S. application Ser. No. 15/234,692, recently filed by the inventor may be utilized.

As further illustrated by FIG. 5, upon answering the questions and conducting the vision-related tests presented to a user 410, a server 430 then receives the user-generated answers and test results. A processor 510 then processes the user-generated answers and test results and compares the user's 410 answers and test results to each vision-related symptom and test result stored within the database 440 to establish a statistical correlation between the user-generated answers and test results to the vision-related symptoms and test results stored within the database 440. Although FIG. 5 illustrates a processor 510 as a component of the system 400 separate and apart from a server 430, it is understood that the present disclosure contemplates embodiments where a server 430 possesses the same capabilities and functionalities as a processor 510. For example, in some preferred embodiments of the present disclosure, a server 430 alone may process and compare the user's 410 answers and test results to establish such a statistical correlation with the vision-related symptoms and test results stored within the database 440, therefore eliminating the need for a separate or stand-alone processor 510 within the system 400 in such embodiments.

Once a statistical correlation is established between a user's 410 answers and test results and the vision-related symptoms and test results within the system's 400 database 440, a server 420 then communicates eye-care provider data 452, alert data 453, field of medicine data 454, and subfield of medicine data 455, if any, that statistically correlates to the vision-related symptoms within the vision-related symptom data 450 and test results within test result data 460 stored in the system's 400 database 440. A server 430 then generates output information including any eye-care provider data 452, alert data 453, field of medicine data 454, and subfield of medicine data, if any, that is tied to the vision-related symptom data 450 and test result data 460 which have a statistical correlation to a user's answers and test results. A server 430 then provides such output information to a user 410 via a user interface 420.

It is understood that the present disclosure contemplates all embodiments where any combination of eye-care provider data 452, alert data 453, field of medicine data 454, or subfield of medicine data may be tied to the vision-related symptoms stored within the vision-related symptom data 450 and test results stored within the test result data 460 and, therefore, included within the output information generated and provided to a user 410 via a user interface 420. For example, in one embodiment, only field of medicine data 454 and subfield of medicine data 455 is tied to the vision-related symptoms contained within the vision-related symptom data 450 and test results contained within the test result data 460 and, therefore, is the only data communicated, generated, and provided by the server 430 as output information. In another example, in one preferred embodiment, only eye-care provider data 452 is tied to the vision related symptoms within the vision-related symptom data 450 and test results within the test result data 460, and, in turn, is the only data communicated, generated, and provided by the server 430.

In some embodiments of the present disclosure, the output information generated and provided to the user 410 via a user interface 420 may be limited based on the statistical correlation level or percentage between the answers and test results generated and the vision-related symptoms and test results stored within the system 400 database 440. For example, in one potential embodiment, only data tied to the vision-related symptoms within the vision-related symptom data 450 and test results within the test result data 460 which have a sixty percent or greater statistical correlation to a user's 410 answers and test results may be generated and provided as output information by a server 430 to a user 410. In other preferred embodiments the limiting statistical correlation percentage may be a seventy percent or greater, an eighty percent or greater, a ninety percent or greater, etc. statistical correlation between the vision-related symptoms within the vision-related symptom data 450 and the test results within the test result data 460. Additionally, the present disclosure contemplates embodiments where all data tied to the vision-related symptoms within the vision-related data 450 and test results within the test result data 460 are communicated, generated, and provided to a user 410 by a server 430, regardless of the statistical correlation level or percentage. Thus, in one preferred embodiment, the output information provided to a user 410 via a server 430 and user interface 420 contains information embedded within the data tied to the vision-related symptom data 450 and test result data 460 stored within the system 400 database 440 as well as the statistical correlation level or percentage in which such data has with the answers and test results generated by a user 410.

In one preferred embodiment of the present disclosure, the eye-care provider data 452 communicated, generated, and provided to a user 410 via a server 430 is limited by a processor 510 based on distance between user's 410 location and eye-care provider professional addresses stored within the eye-care provider data 452. In such embodiments, a user's 410 location may be determined through the use of a global positioning system receiver module 370 present at the device, as illustrated in FIG. 3, used by a user 410 to access the system database 440. In this embodiment, a server 430 receives and communicates to a processor 510 a user's 410 location. Once a processor 510 receives such communication the processor 510 limits the eye-care provider data generated by a server 430 and provided to a user 410 in the output information by calculating the distance between the user's location 410 and the location of each pertinent eye-care provider's address stored within database 440 as eye-care provider data 452. For example, in one embodiment, only eye-care provider data 452 which relates to eye-care providers within ten miles of a user's 410 location may be communicated, generated, and provided to a user 410 by a server 420.

It is understood that devices used by a user 410 to access the system 400 database 440 may include, but is not limited to: desktop computers, laptops, tablets, and mobile phone devices. Moreover, it is understood that the present disclosure contemplates all embodiments where the distance that limits the output information provided to a user 410 is self-contained and solely determined by system administrators of the system 400, embodiments where a user 410 selects such distance by allowing a user 410 to personally apply a filter via a user interface 420, or any blend or combination thereof. For example, in a blended embodiment, the limiting distance may be initially determined by a system administrator of the system 400, but the system 400 allows a user 410 to pay a fee to either increase or decrease the distance which limits the output information communicated, generated, and provided by a server 430, and upon receipt of such payment a system administrator manually or the system 400 itself automatically sets the distance to the user's 410 desired distance.

In yet another preferred embodiment, the eye-care provider data 452 communicated, generated, and provided to a user 410 via a server 430 is limited by a processor 510 based on a series of geographical parameters stored within the eye-care provider data 452 that relates to the eye-care providers stored within the eye-care provider data 452. Such geographic parameters may include, but are not limited to: the state in which a specific eye-care provider is located, the city in which a specific eye-care provider is located, the town in which a specific eye-care provider is located, the neighborhood in which a specific eye-care provider is located, and whether the eye care provider is near to a specific location or area designated by a user or system administrator. In such embodiments, a user's 410 location may be determined through the use of a global positioning system receiver module 370 present at the device, as illustrated in FIG. 3, used by a user 410 to access the system database 440. In this embodiment, a server 430 receives and communicates to a processor 510 a user's 410 location. Once a processor 510 receives such communication a processor 510 limits the eye-care provider data generated and provided by a server 430 to a user 410 by determining whether the eye-care provider data 453 which corresponds to the vision-related symptoms data 450 and test result data 460 correlating to a user's 410 answers and test results also matches the limiting geographic parameters set by a system administrator or selected by a user 410. The present disclosure contemplates all embodiments where the geographic parameters which limit the output information provided to a user 410 is self-contained and solely determined by system administrators of the system 400, embodiments where a user 410 personally applies a geographic parameters filter utilizing a user interface 420, or any blend or combination thereof.

In one preferred embodiment, the eye-care provider data 452 communicated, generated, and provided to a user 410 via a server 430 is limited by a processor 510 based on whether or not the eye-care providers within the eye-care provider data 452 that is tied to the vision-related symptom data 450 and test result data 460 which correlate to a user's 410 answers and test results is open on Saturdays or provides other special accommodations for patients. Again, the present disclosure contemplates all embodiments where the open on Saturday or other special accommodations limitation is self-contained and solely determined by a system administrator of the system 400, embodiments where a user 410 personally applies a patient accommodation filter utilizing a user interface 420, or any blend or combination thereof.

In another preferred embodiment, the system 400 and system database 440 is accessible to a user 410 via a mobile application, wherein the mobile application is in communication with the database 440 via a server 430. In such an embodiment, a mobile device serves as a user interface 420 and a mobile application contained within the mobile device displays the output information generated and provided by a server 430 from the database 440. In yet another preferred embodiment of the present disclosure, the output information generated and provided by a server 430 is provided to a user interface 420 via email. In this embodiment, a user 410 navigates through a web browser to the user's 410 email provider in order to access the output information generated by the system 400.

FIG. 7 illustrates a representation of an example architecture of how the system 400 determines whether an eye-care provider 730 may access or manipulate the eye-care provider data 710 corresponding to the eye-care provider 730 requesting access to the database 440. As illustrated in FIGS. 4 and 7, In addition to a user interface 420, in some preferred embodiments of the present disclosure the system 400 further comprises an eye-care provider user interface that communicates with the database 440 via a server 430. The eye-care provider user interface implemented within the system 400 enables physicians, specialists, or any other individual or organizational entity stored within the eye-care provider data 452 as an eye-care provider 730 to access and manipulate the eye-care provider data 710 that specifically relates to the requesting eye-care provider 730. For example, through the eye-care provider interface an eye-care provider 730 may access the data within the eye-care provider data 452 that corresponds to the eye-care provider's professional name and address, make edits or change such information, and store such changes to the database 440. To gain such access and the ability to make such manipulations, an eye-care provider first makes a request to a server 430 to access the eye-care provider data 452 within the database 440.

As illustrated in FIG. 7, to ensure eye-care providers can only access and manipulate the eye-care provider data 710 that specifically relates to the requesting eye-care provider 730, if any, each eye-care provider 730 has a certain permission level 720 that dictates whether an eye-care provider 730 has the ability to access and manipulate such data. In one preferred embodiment, an eye-care provider's 730 permission level 720 corresponds to an eye-care provider account within the system database 440. In one embodiment, an eye-care provider 730 establishes a system account by communicating its email address and user-created password to the database 440 of the system 400 via a server 430 and storing such information within the database 440. In another embodiment, an eye-care provider 730 may establish an eye-care provider system account by creating an individualized username and user-created password to the database of the system 400 via a server and storing such information with the database 440.

Upon the establishment of an eye-care provider system account, either a system administrator manually or the system 400 itself automatically may assign specific permission levels to each eye-care provider system account. For example, upon the creation of an eye-care provider system account, a system administrator or the system 400 itself may authorize an eye-care provider 730 to access and manipulate the name, address, and telephone number of the eye-care provider 730 as stored within the eye-care provider data 452 in the database 440.

As illustrated in FIGS. 4 and 7, upon receiving an eye-care provider's request to access the database 440 of the system, a server 430 then verifies the eye-care provider's permission level 720 to access or manipulate the eye-care provider data 710 that corresponds to that eye-care provider 730. If the eye-care provider requesting access to the database does not have a sufficient or valid permission level 720, a server 430 will deny the request and the eye-care provider 730 will not be permitted to access or manipulate any data within the eye-care provider data 452. If the eye-care provider 730 requesting access does have a sufficient or valid permission level 720, a server 430 will receive the eye-care provider data 710 relating to the requesting eye-care provider's 730 request within the eye-care provider data 452 in the database 440. A server 430 will then communicate the eye-care provider data 710 associated with the requesting eye-care provider 730, based on the eye-care provider's 730 request. Following this communication a server 430 will then generate the eye-care provider data 710 corresponding to the requesting eye-care provider 730 and will subsequently provide such eye-care provider data 710 to the requesting eye-care provider 730 via a user interface 420.

The system 400 will then allow the eye-care provider 730 to access or manipulate the eye-care provider data 710 provided via a user interface 420. In the event that the eye-care provider manipulates or edits the eye-care data 452 provided in the previous steps, a processor 510 will process such changes or edits and a server 430 will subsequently store the changes or edits in the database 440.

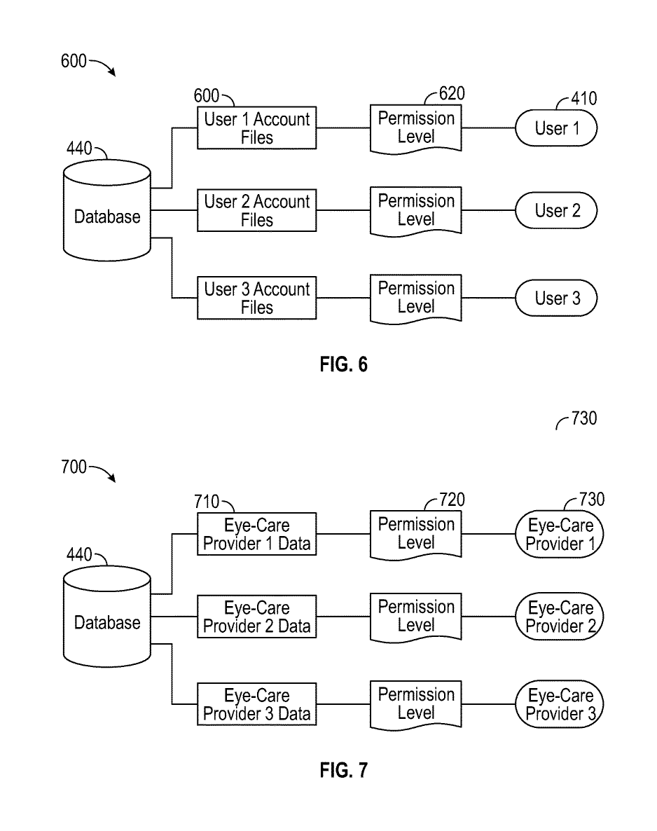

FIG. 6 illustrates a representation of an example architecture of how the system 400 stores user account files 610 and determines whether a user 410 may access or manipulate user account files 610 corresponding to a requesting user 410. Nonetheless, it is not required that a user account be created for a user to utilize the system set forth herein. In one embodiment, without creating a user account, a user may navigate to a website, answer a series of questions, complete at least one vision-related test, and receive information and eye-care provider information, as set forth herein. In this embodiment, the user may be asked to create a user account upon completing the questions and answers and receiving information and eye-care provider. Preferably, the user will be prompted to create an account so the user's information can be stored with the user's personal account and compared for future testing.

As illustrated in FIGS. 4 and 6, in one preferred embodiment of the present disclosure, a series of user account files 610 fields are stored within the system's database 440. Within this embodiment, each user account file 610 is tied to a specific user 410 such that the user account files 610 relating a specific user 410 are grouped with that user 410. Each user 410 has the option of having zero or more user account files 610 tied to the user 410. In one preferred embodiment a user's 410 user account files 610 are tied to user 410 through the use of a user account. In one embodiment, a user 410 establishes a system account by communicating its email address and user-created password to the database 440 of the system 400 via a server 430 and storing such information within the database 440. To establish a user account within the system, a user 410 may provide and store his email address and user-created password to the system's database 440 by utilizing a user interface 420 and server 430. Alternatively, a user account may be established by a user providing and storing a user-created username and password within the system's database 440 through the use of a user interface 420 and server 430.

A file stored within a user account file 610 may include, but is not limited to: answers and test results generated by a user 410; the output information communicated, generated, and provided to a user 410 by the server based on the statistical correlation between user-generated answers and test results and the vision-related symptom data 450 and test result data 460 within the database 440, as discussed above; or any other possible information generated by the system or stored within the system's database 440. User-generated answers and test results are stored within a user account by a server 430 first communicating such answers and test results to the user's 410 corresponding account and subsequently storing such information within the user's 410 account. The output information communicated, generated, and provided to a user 410 by the server based on the statistical correlation between user-generated answers and test results and the vision related symptom data 450 and test result data 460 are stored within a user account by a server 430 first communicating such output information to the user's 410 corresponding account and then subsequently storing such output information to the user's 410 account.

In order for a user 410 to access the user account files 610 associated with the user's 410 account, a user must first make a user request to a server 430 to access or manipulate the user's account files 610 within the database 440. To ensure a user 410 can only access and manipulate user account files 610 data that are specifically tied to the requesting user 410, if any, each user 410 has a permission level 620 with the user 410 that dictates whether the user 410 has the ability to access and manipulate such data. A user's 410 permission level 620 is established and configured in the same or similar fashion as the eye-care provider permission levels 710 discussed above, except that user permission levels 620 correspond to user account files 610 instead of eye-care care provider data. A server 430 verifies a user's 410 permission level 620 and permits or denies a user's 410 request to access user account files 610 based on the user's 410 permission level 620. If a requesting user 410 has a sufficient or valid permission level to access the requested user account files 610, a server 430 will then receive the requested user account files 610 from the database 440, based on a user's request and permission level. After receiving such account files 610 from the database 440, a server 430 will then communicate, generate, and provide the account files 610 based on the user's 410 request to the user 410 via a user interface 420.

In one preferred embodiment of the present disclosure, a user 410 may compare prior answers and test results stored within the user's 410 user account files 610 with new answers and test results to determine the progression of a user's 410 condition or to determine whether a user 410 needs to promptly see an eye-care provider. In such an embodiment, alert data 453 indicating the progression of a user's 410 condition or whether the user 410 needs to promptly see an eye-care provider is stored within the database 440 and tied to statistical variances 456 indicative of differences between various eye-related symptoms and differences between various test results stored within the database 440. A server 430 receives the most recent answers and test results stored within a user's 410 user account files 610 and communicates such answers and test results, wherein these answers and test results serve as the baseline in which new answers and test results are compared. To generate new answers and test results (reflecting the present state of the user's 410 condition), the user 410 repeats certain steps as disclosed above. Once a new set of answers and test results are generated, such answers and test results are received and communicated by a server 430. A processor 510 then processes both the previously stored answers and test results and the new set of answers and test results to establish a statistical variance, if any, between the previously stored answers and test results and the new set of answers and test results generated by the user 410. Alert data 453 corresponding to the statistical variance established between the previously stored answers and test results and the new set of answers and test results, if any, is received and communicated by a server 430. A server 430 then generates output information that includes the alert data 453 corresponding to the statistical variance discussed above, if any, and ultimately provides to the user 410 such output information via a user interface 420. In one preferred embodiment, a server 430 may communicate, generate, and ultimately provide such output information to an eye-care provider 730 having a sufficient permission level 720 via an eye-care provider user interface in accordance with the disclosure below.

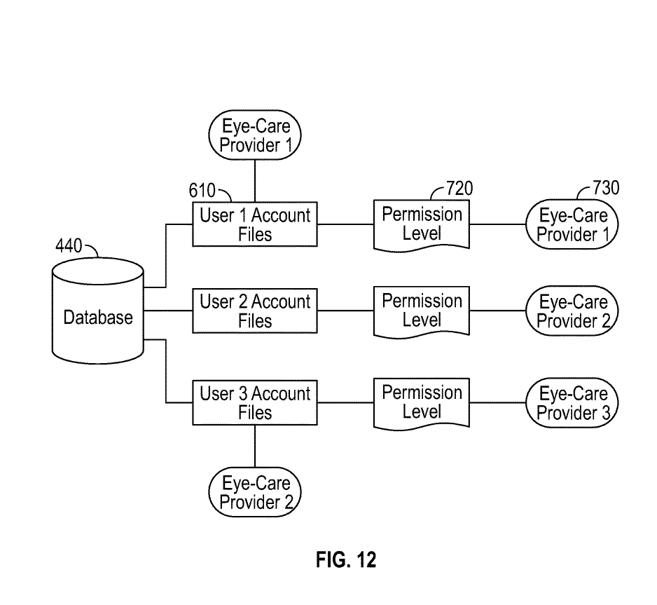

As illustrated in FIG. 12, in another preferred embodiment, eye-care providers 730 may be able to access user account files 610 depending on the eye-care provider's 730 permission level 720. In such embodiments, user account files 610 are stored within the database 440 and each user account files 610 is tied to zero or more eye-care providers 730 within the eye-care provider data 452.

Such embodiments utilize an eye-care provider user interface that is in communication with the database 440 via a server 430. To access user account files 610 within the database 440, an eye-care provider 730 must make such a request to a server 430 via the eye-care provider user interface. Each eye-care provider 730 has certain permission levels 720 that allow each eye-care provider 730 to access user account files 610 that are tied to the eye-care provider 730, if any. Preferably, a user's 410 user account files 610 are tied to an eye-care provider 730 by a user 410 granting permission for a specific eye-care provider 730 to access the user's 410 user account files 610. A server 430 will verify an eye-care provider's 730 permission level 720 to access user account files 610 tied to the eye-care provider 730, if any, and will grant or deny the eye-care provider's 730 request based on the eye-care provider's 730 permission level 720. Thus, each eye-care provider's 730 ability to access user account files 610 is limited based on the eye-care provider's 730 permission level 720. If the eye-care provider's 730 request is granted, a server 430 will receive, communicate, generate, and ultimately provide to the eye-care provider 730 user account files 610 that are tied to the eye-care provider 730 via the eye-care provider user interface.

Yet another embodiment of the present disclosure comprises a user interface, a server, a processor, a database, and a non-transitory computer-readable medium for identifying an appropriate eye-care provider. In a preferred embodiment, the non-transitory computer-readable medium is coupled to the processor and server and has instructions stored thereon, which when executed by the processor, cause the processor to perform certain operations. In a preferred implementation, the operations may comprise, receiving, form a user interface 420, a request to access and compare vision-related symptoms data 450 and test result data 460 stored within the database 440 to vision-related symptoms experienced by a user 410. Furthermore, the operations may comprise the server 430 receiving a series of questions and at least one vision-related test, wherein such questions and vision-related tests correlate to the vision-related symptom data 450 and test result data 460 stored within the database 440. The operations may further comprise, the server 430 communicating, generating, and providing such series of questions and vision-related tests stored within the database 440 to a user 410 via the user interface 420. The operations may further comprise, allowing a user 410 to input or select answers to the series of questions and conduct such vision-related tests communicated, generated, and provided by the server 430. The operations may further comprise, the server 430 receiving the answers and test results generated by a user's 410 with the user interface 420. The operations may further comprise, the processor 510 processing the answers and test results generated by a user's interaction with the user interface 420 to establish a statistical correlation between user-generated answers and test results to vision-related symptoms data 450 and test result data 460 stored within the database 440. The operations may further comprise, the server 430 communicating informative data tied to vision-related symptom data 450 and test result data 460 stored within the database 440 which has a statistical correlation to answers and test results generated by a user 410. The operations may further comprise, the server 430 generating output information including informative data tied to vision-related symptom data 450 and test result data 460 stored within the database 440 which has a statistical correlation to answers and test results generated by a user 410. The operations may further comprise, providing, by the server 430 to the user interface 430, such output information.

In a preferred embodiment, the system is written in C# and utilizes Microsoft ASP.NET. However, it is understood that embodiments of the invention disclosed herein may be created from any language using any framework. In one implementation the system comprises a suite of web services that power all of the applications and tools that comprise the system. For example, web-based management tools, as well as the web-based delivery application, both interface with the same set of services.

In one embodiment the system utilizes a database management system such as Microsoft SQL or similar. It is understood that various servers may be used to access stored data. Users must connect through a service or server before accessing stored data. This ensures all access to the database has been authenticated and restricts users to changes within their organization or entity, as set forth herein.

In a preferred embodiment, a user uses a web browser to navigate to a system website. The system website comprises a plurality of web pages. In a preferred embodiment, each webpage is accessed via tabs on the system website homepage or other system webpage. Links or tabs allow a user to navigate from one page to another. Implementations of the invention further comprise web-based forms with text fields therein. In a preferred embodiment, said text fields auto populate predetermined forms, webpages, databases, servers, or other targeted destinations. In one embodiment, a user accesses a webpage that accesses a database via a server.

The system and method set forth herein may find utility in many areas in which identifying an appropriate eye-care provider is desired. The system may find particular utility within the field of ophthalmology. For example, a user experiencing blurred vision may desire to seek out treatment for her condition, but is unsure as to what eye-care provider to seek treatment from. However, by utilizing the system and method disclosed herein, the user can readily make such a determination. The user will first request access to the database of the system. A server will then receive, communicate, and provide a series of questions and at least one test to the user, based on the user's request. Upon answering these questions and conducting the tests provided, the user's answers and test results will then be received, communicated, and processed by the system to establish a statistical correlation between the symptoms and test results stored within the system's database and the user's experienced symptoms. Based on the presence of a statistical correlation, the system will return output information to the user containing one or more fields of information indicating what type of eye-care provider the user statistically most likely needs treatment from. Accordingly, if the user's experienced symptoms are statistically consistent with age-related macular degeneration, the output information presented by the system may indicate that the user should seek treatment from a retina specialist.

It will also be apparent to one of ordinary skill in the art that aspects of the present disclosure, as described above, may be implemented in many different forms of software, firmware, and hardware in the implementations illustrated in the figures. The actual software code or specialized hardware used to implement aspects consistent with the present disclosure is not limiting of the present invention. Thus, the operation and behavior of the aspects where described without references to the specific software code—it being understood that one of ordinary skill in the art would be able to design software and hardware to implement the aspects based on the description herein.

What is claimed is:

1. A method of identifying an appropriate eye-care provider, said method comprising the steps of:
a. Storing vision-related symptoms and test result data within a database;
i. Said vision-related symptoms being tied to a series of questions and possible answers which correlate to each said vision-related symptom;
ii. Said test result data being tied to one or more tests which correlate to each test result within the test result data;
iii. Said vision-related symptoms and test result data being tied to a specific field of medicine such that said vision-related symptoms and test result data relating to said field of medicine are grouped with said field of medicine;
iv. Each field of medicine having an option of having zero or more symptoms and test result data tied to said field of medicine;
v. Each field of medicine having zero or more subfields of medicine concerning specific practice areas within each said field of medicine to which said vision-related symptoms and test result data stored within said database may relate;
b. Requesting, by a user, a server via a user interface to access and compare said vision-related symptoms and test result data contained within said database to said user's experienced symptoms;
c. Receiving, by a server, said series of questions and at least one vision-related test correlating to said vision-related symptoms and test result data stored within said database;
d. Communicating, by the server, based on the received user request, said series of questions and vision-related tests;
e. Generating, by the server, said series of questions and said vision-related test;
f. Providing, by the server to the user, said series of questions and vision-related test to a user interface;
g. Allowing said user to input or select answers to the series of questions and conduct the vision-related test generated by the server and presented to the user via said user interface;
h. Receiving, by the server, the user-generated answers and test results;
i. Processing, by a processor, the user-generated answers and test results to establish a statistical correlation between the user-generated answers and test results to each vision-related symptom and test result stored within said database;
j. Communicating, by the server, zero or more fields and subfields of medicine tied to the vision-related symptoms and test results stored within said database which have a statistical correlation to the user-generated answers and test results;
k. Generating, by the server, output information that includes the fields and subfields of medicine tied to the vision-related symptoms and test results within said database with the statistical correlation to the user-generated answers and test results; and
l. Providing, by the server to the user, said output information via a user interface.

2. The method of claim 1, further comprising:
a. Storing eye-care provider data within said database;
i. Said eye-care provider data being tied to a specific field or subfield of medicine such that said eye-care provider data relating to said field of or subfield of medicine is grouped with said field or subfield of medicine;
ii. Said eye-care provider data containing the information regarding eye-care providers within the field or subfield of medicine in which the eye-care provider data is tied;
b. Communicating, by the server, said eye-care provider data tied to the fields and subfields of medicine communicated within step (j) of claim 1;
c. Generating, by the server, said output information including said eye-care provider data tied to the fields and subfields of medicine generated in step (k) of claim 1; and
d. Providing, by the server to the user, said output information to a user interface.

3. The method of claim 2, further comprising:
a. Communicating, by the server, the user's location;
b. The location of the user being determined by a global positioning system receiver module at the device used by the user to access said database;
c. Limiting, by the processor, the eye-care provider data generated by the server for said output information based on the distance between the user's location and each eye-care provider's professional address.

4. The method of claim 2, further comprising:
a. Said eye-care provider data having specific geographic parameters related thereto;
b. The location of said specific geographic parameters being determined by a global positioning system;
c. Limiting, by the processor, the eye-care provider data generated by the server for said output information based on said specific geographic parameters.

5. The method of claim 2, further comprising:
a. Said eye-care provider data indicating whether the eye-care provider is open on Saturdays or provides other special accommodations for patients, if applicable;
b. Communicating, by the server, said eye-care provider data;
c. Limiting, by the processor, said eye-care provider data generated by the server for said output information based on whether the eye-care provider is open on Saturdays or provides other special accommodations for patients.

6. The method of claim 1, further comprising:
a. Storing alert data within said database;
i. Said alert data being tied to said vision-related symptoms and test result data such that said alert data relating to said symptoms and test result data are grouped with said symptoms and test result data;
ii. Said alert data indicating the urgency, severity, or progression of said user's condition;
b. Communicating, by the server, said alert data based on the strength of the statistical correlation between the user-generated answers and test results and the vision-related symptoms and test result data stored within said database.
c. Generating, by the server, said output information including said alert data; and
d. Providing, by the server to the user, said output information including said alert data to said user interface.

7. The method of claim 1, further comprising:
a. Said user interface being accessible to said user via a mobile application;
b. Said mobile application being in communication with the database via a server; and
c. Said mobile application displaying the output information generated by the server from the database on the user interface.

8. The method of claim 1, further comprising:
a. Said output information, provided by the server to the user interface, being delivered via email;
b. Said user navigating, via web browser, to said user's email provider to access said output information.

9. The method of claim 2, further comprising:
a. An eye-care provider user interface being in communication with the database via a server;
b. Requesting by an eye-care provider, a server via the eye-care provider user interface to access said eye-care provider data within said database;
i. Said eye-care providers having certain permission levels that allow each eye care provider to access or edit certain aspects of the eye-care provider data;
ii. Said server verifying each eye-care provider's permission level to access or manipulate said eye-care provider data;
iii. Each eye care provider's ability to access or edit said eye-care provider data being limited by the permission level of said eye-care provider;
c. Receiving, by a server, said eye-care provider data stored within said database, based on the eye-care provider's request and permission level;
d. Communicating, by the server, based on the received eye-care provider request said eye-care provider data;
e. Generating, by the server, said eye-care provider data; and
f. Providing, by the server, to the eye-care provider via the eye-care provider user interface said eye-care provider data;
g. Allowing said eye-care provider to access or edit said eye-care provider data via the eye-care provider user interface;
h. Processing, by the processor, said eye-care provider's edits to said eye-care provider data; and
i. Storing, by the server, said eye-care provider's edits to said eye-care provider data to the database.

10. A method of identifying an appropriate eye-care provider said method comprising the steps of:
a. Storing vision-related symptoms and test result data within a database;
i. Said vision-related symptoms being tied to a series of questions and possible answers which correlate to said vision-related symptoms;
ii. Said vision-related symptoms and test result data being tied to eye-care provider data such that said vision-related symptoms and test result data relating, if any, to said eye-care provider data are grouped with said eye-care provider data;
iii. Said eye-care provider data including information regarding eye-care providers who treat eye-related defects associated with the vision-related symptoms and test results stored within said database;
b. Requesting, by a user, a server via a user interface to access and compare said vision-related symptoms and test result data contained within said database to said user's experienced symptoms;
c. Receiving, by a server, said series of questions and at least one vision-related test correlating to said vision-related symptoms and test result data stored within said database;
d. Communicating, by the server, based on the received user request said series of questions and vision-related tests;
e. Generating by the server, said series of questions and vision-related test;
f. Providing, by the server to the user, said series of questions and vision-related test to a user interface relating to the user's request and permission level;
g. Allowing said user to input or select answers to said series of questions and conduct the vision-related test generated by the server and presented to the user via said user interface;
h. Receiving, by the server, the answers and test results generated by the user;
i. Processing, by a processor, the answers and test results generated by the user to establish a statistical correlation between the answers and test results generated by the user to the vision-related symptom and test result data stored within said database;
j. Communicating, by the server, eye-care provider data tied to the vision-related symptoms and test results stored within said database which have a statistical correlation to the answers and test results generated by the user;
k. Generating, by the server, output information that includes the eye-care provider data tied to the vision-related symptom and test result data with a statistical correlation to the answers and test results generated by said user; and
l. Providing, by the server, to the user, said output information via a user interface.

11. The method of claim 10, further comprising:
a. Said eye-care provider data having specific geographic parameters related thereto;
b. The location of said geographic parameters being determined by a global positioning system;
c. Limiting, by the processor, the eye-care provider data generated by the server for said output information based on said specific geographic parameters.

12. The method of claim 10, further comprising:
a. Allowing the user to apply a geographical filter via the user interface;
b. Said geographical filter limiting the eye-care provider data contained within the output information generated by the server based on the distance between the user's location and the eye-care provider's office;
c. Communicating, by the server, the user's location;
d. The location of the user being determined by a global positioning system receiver module located within the device used by the user to access the database; and
e. Limiting, by the processor, the eye-care provider data generated by the server within the output information based on the geographical filter selected by the user and the distance between the user's location and the eye-care provider's office.

13. The method of claim 10, further comprising:
a. Allowing the user to apply a patient accommodation filter via the user interface;
b. Said patient accommodation filter limiting the eye-care provider data contained within the output information generated by the server based on whether an eye-care provider is open on Saturdays or provides other patient accommodations;
c. Communicating, by the server, the patient accommodation filter selected by the user;
d. Limiting, by the processor, the eye-care provider data generated by the server within the output information based on the patient accommodation filter selected by the user.

14. The method of claim 10, further comprising:
a. Storing alert data within said database;
i. Said alert data being tied to said vision-related symptoms and test result data such that said alert data relating to said symptoms and test result data are grouped with said symptoms and test result data;
ii. Said alert data indicating the urgency, severity, or progression of said user's condition;
b. Communicating, by the server, said alert data based on the statistical correlation between the user-generated answers and test results and the vision-related symptoms and test result data stored within said database;
c. Generating by the server, said output information including said alert data; and
d. Providing, by the server to the user, said output information including said alert data to said user interface.

15. The method of claim 10, further comprising:
a. Said user interface is accessible to said user via a mobile application;
b. Said mobile application being in communication with the database via a server; and
c. Said mobile application displaying the output information generated by the server from the database on the user interface.

16. The method of claim 10, further comprising:
a. Said output information, provided by the server to the user interface, being delivered via email;
b. Said user navigating, via web browser, to said user's email provider to access said output information.

17. The method of claim 10, further comprising:
a. Storing user account files within said database;
i. Said user account files being tied to a specific user such that said user account files relating to said user are grouped with said user;
ii. Each user having an option of having zero or more user account files tied to said user;
iii. Said users having certain permission levels that allow each user to access or edit certain files and data relating to said user;
b. Communicating, by the server, the answers and test results generated by the user to the user's account file within said database;
c. Storing the answers and test results generated by the user in the user's account file within said database;
d. Communicating, by the server, said output information generated in step (k) of claim 10 to the user's account file within said database; and
e. Storing said output information generated in step (k) of claim 10 within the user's account file within said database.

18. The method of claim 17 further comprising:
a. Storing alert data within said database;
i. Said alert data being tied to statistical variances indicative of differences between various eye-related symptoms and differences between various test results stored within the database;
ii. Said alert data indicating the progression of a user's condition or whether a user needs to promptly see an eye-care provider;
b. Receiving, by a server, the most recent answers and test results stored within a user's account file;
c. Communicating, by a server, the most recent answers and test results stored within a user's account file;
d. Repeating steps (b) through (h) of claim 10 such that a new set of answers and test results are generated by the user and received by a server;
e. Processing, by a processor, the answers and test results of step (c) and step (d) to establish any statistical variance, if any, between the answers and test results of step (c) and those received by a server in step (d);
f. Receiving, by a server, alert data stored within the database corresponding to the statistical variance established in step (e), if any;
g. Communicating, by a server, alert data stored within the database corresponding to the statistical variance established in step (e), if any;
h. Generating, by a server, output information that includes the alert data corresponding to the statistical variance established in step (e), if any;
i. Providing, by a server, to the user, said output information via a user interface.

19. The method of claim 17, further comprising:
a. Requesting, by a user, a server to access or edit the user's user account file stored within said database;
i. Said server verifying the user's permission level to access or edit said user account file;
ii. The access to the user's account file being limited by the permission level of said user;
b. Receiving, by a server, requested user account file from the database based on a user's request and permission level;
c. Communicating, by the server, based on the received user request, the requested user account file;
d. Generating, by the server, requested user account file; and
e. Providing, by the server to the user, said user account file to a user interface relating to the user's request and permission level.

20. The method of claim 10, further comprising:
a. An eye-care provider user interface being in communication with the database via a server;
b. Requesting, by an eye-care provider, a server via the eye-care provider user interface to access said eye-care provider data within said database;
i. Said eye-care providers having certain permission levels that allow each eye care provider to access or edit certain aspects of the eye-care provider data;
ii. Said server verifying each eye-care provider's permission level to access or edit said eye-care provider data;
iii. Each eye care provider's ability to access or edit said eye-care provider data being limited by the permission level of said eye-care provider;
c. Receiving, by a server, said eye-care provider data stored within said database;
d. Communicating, by the server, based on the received eye-care provider request and eye-care provider permission level, said eye-care provider data;
e. Generating, by the server, to the eye-care provider via the eye-care provider user interface said eye-care provider data;
f. Allowing said eye-care provider to edit said eye-care provider data via the eye-care provider user interface;
g. Processing, by the processor, said eye-care provider's edits, if any, to said eye-care provider data, and;
h. Storing, by the server, said eye-care provider's edits, if any, to said eye-care provider data to the database.

21. The method of claim 10, further comprising:
a. Storing user account files within said database;
i. Said user account files being tied to a specific user such that said user account files relating to said user are grouped with said user;
ii. Each user having an option of having zero or more user account files tied to said user;
iii. Said user account files being further tied to zero or more eye-care providers within the eye-care provider data;
b. An eye-care provider user interface being in communication with the database via a server;
c. Requesting, by an eye-care provider, a server via the eye-care provider user interface to access said user account files within said database;
i. Said eye-care providers having certain permission levels that allow each eye-care provider to access user account files that are tied to the eye-care provider, if any;
ii. Said server verifying each eye-care provider's permission level to access user account files tied to the eye-care provider, if any;
iii. Each eye care provider's ability to access user account files being limited by the permission level of said eye-care provider;
d. Receiving, by a server, said user account files that are tied to the requesting eye-care provider;
e. Communicating, by the server, based on the received eye-care provider request and eye-care provider permission level, said user account files;
f. Generating, by the server, to the eye-care provider via the eye-care provider user interface said user account files; and
g. Providing, by a server, to an eye-care provider via the eye-care provider user interface said user account files.

22. The method of claim 21, further comprising:
a. Storing alert data within said database;
i. Said alert data being tied to statistical variances between various eye-related symptoms and between various test results stored within the database;
ii. Said alert data indicating the progression of a user's condition or whether a user needs to promptly see an eye-care provider;
b. Processing, by a processor, the answers and test result data stored within a user's account file to establish a statistical variance, if any, between prior answers and test results generated by a user and the most recent answers and test results generated by a user;
c. Receiving, by a server, alert data stored within the database corresponding to the statistical variance established in step (b), if any;
d. Communicating, by a server, alert data stored within the database corresponding to the statistical variance established in step (b), if any;
e. Generating, by a server, output information that includes alert data corresponding to the statistical variance established in step (b), if any; and
f. Providing, by a server, to the eye-care provider, said output information via the eye-care provider user interface.

23. A system comprising:
a. a user interface;
b. a server;
c. a processor;
d. a database; and
e. a non-transitory computer-readable medium coupled to the processor and the server having instructions stored thereon, which, when executed by the processor, cause the processor to perform operations comprising:
i. Receiving, from the user interface, a request to access and compare vision-related symptoms and test result data stored within the database to vision-related symptoms experienced by a user;
ii. Receiving, by the server, a series of questions and at least one vision-related test, wherein such questions and vision-related tests correlate to the vision-related symptoms and test result data stored within the database;
iii. Communicating, by the server, such series of questions and vision-related tests stored within said database;
iv. Generating, through the server, such series of questions and vision-related tests to the user interface;
v. Providing, through the server, such series of questions and vision-related tests to the user interface;
vi. Allowing a user to input or select answers to such series of questions and conduct such vision-related tests generated by a server via the user interface;
vii. Receiving, by the server, answers and test results generated by a user's interaction with the user interface;
viii. Processing, by the processor, the answers and test results generated by a user's interaction with the user interface to establish a statistical correlation between user-generated answers and test results to vision-related symptoms and test result data stored within the database;
ix. Communicating, through the server, informative data tied to vision-related symptoms and test result data stored within the database which has a statistical correlation to answers and test results generated by a user;
x. Generating, by the server, output information including informative data tied to vision-related symptoms and test result data stored within the database which has a statistical correlation to answers and test results generated by a user; and
xi. Providing, by the server to the user interface, such output information.
